(19) United States
(12) Patent Application Publication
(10) Pub. No.: US 2016/0375224 A1
(43) Pub. Date: Dec. 29, 2016

(54) KIT FOR HOLDING AND PROTECTING CATHETERS THAT ARE PLACED ON AN AREA OF THE BODY OF A PATIENT

(71) Applicant: P & P INNOVATION (SARL), Cassis (FR)

(72) Inventor: Nathalie GILLI, Les Brayes (FR)

(21) Appl. No.: 15/125,908

(22) PCT Filed: Mar. 16, 2015
(86) PCT No.: PCT/FR2015/050627

§ 371 Date: Sep. 13, 2016

(30) Foreign Application Priority Data
Mar. 17, 2014 (FR) 14 52187

(51) Int. Cl. A61M 25/02

(52) U.S. Cl. CPC A61M 25/02; A61M 2025/0266; A61M 2025/0246

(57) ABSTRACT

Kit for protecting and holding catheters, characterized in that it comprises, on the one hand, a device (1) having a bearing (3) with an inner face and an outer face, said inner face being intended to be applied to the skin of a patient and having a cavity (3c) shaped to accommodate the non-invasive part (head or connector) of a catheter, while said outer face is attached to the central part of a thin and flexible cover sheet (2) which comprises an unglued protruding peripheral fixing portion extending outwards from said central part, and of which the inner face is intended to be applied to the skin of the patient, and, on the other hand, a package (4) that contains a non-allergenic and water-impermeable adhesive gel, this package having an outlet orifice shaped to allow a string of adhesive gel to be applied to the skin around the placement of the non-invasive part (head or connector) of the catheter, after the latter has been inserted.

The present invention relates to a kit for the maintenance and protection of catheters placed on an area of the body of a patient.

A catheter is a medical device used for a prolonged injection in the body of drugs or solutes or for the withdrawal of fluids (such as blood or pus) of the body. The catheter is comprised of an invasive end composed by a tube of variable widths and flexibilities, inserted in a vessel or a body cavity, by a head or fitting, consisting of the non-invasive section of the catheter and allowing connection with a hose linked to a recovery reservoir or containing a solute or a drug.

To avoid infections and obtain faster healing, it is necessary to change the catheters the least possible. Also, rather than remove and reinsert a new hypodermic needle each time that a drug must be administered, it is often more effective and more comfortable for the patient to insert a needle in the catheter that is then left in place on the skin, sometimes several days and even several months depending on their use.

Once placed, the catheter must move the least possible, more particularly the case when an anaesthetic or post-operative analgesia, implementing perineural catheters.

To prevent excessive movement of the needle, this is generally attached last to the patient.

It is known to use adhesive tape to attach a catheter. This solution having little effect, is often necessary to attach the flexible tubes if extending from the main body of the catheter with the help of adhesive strips bonded onto the skin of the patient.

Such a process of fixing however, is not optimal in that the adhesive elements of fixing are sort of detached so that the catheter does not remain firmly attached to the skin of the patient, particularly if it is positioned at the level of a joint.

To resolve this problem, it has been known to suture the catheter, directly to the skin of the patient. Although this solution is more secure in terms of fixing, it is obviously not preferred by patients and can lead to other complications such as infections.

Devices for easily removable catheters have been developed and are particularly useful in cases where it is necessary to inspect, adjust or replace said catheter.

There also exists a large number of documents disclosing devices and processes according to which a means of retaining a catheter is fixed on a pad, which the lower surface consists of a self-adhesive coating (protected by a removable film) through which wherein the pad can be fixed on the skin of a patient After insertion of the invasive section of catheter in a cavity of the body; such devices and processes are, for example, described in US documents-2007/0.287.963, U.S. Pat. No. 6,117,163, US-2009/0.254.040.

These devices and processes present several disadvantages, for example, they offer an imperfect contact of the self-adhesive backing with the surface of the epidermis, taking into account the reliefs and hollows (wrinkles) that presents said surface, which promotes the delamination of said pad which leads to non-waterproofing around the entrance of the catheter, allowing the passage of water and infectious agents in the direction of the site of implantation of said catheter.

In addition, these devices generally use rigid plastic components with prominent profiles which can be uncomfortable and painful for the patient.

Finally, a major disadvantage of these devices is that the catheters are too exposed, so they are likely to snag and be pulled out compelling the patient or practitioners to constantly pay attention to their movements, which can be uncomfortable.

Devices have been proposed to remedy this disadvantage, particularly with the help of systems to cover the catheter, to avoid contact with external elements that can cause its pulling out.

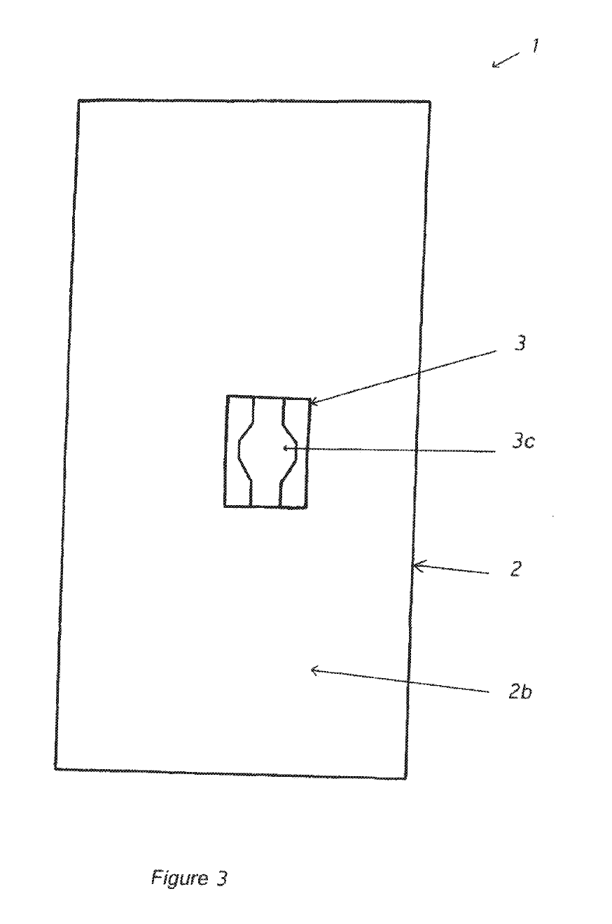

For example, document US-2008/0.065.022 describes (FIG. 3) a catheter fixing device including, on the one hand, a pad with an internal surface intended to be applied on the skin of a patient and an external surface, a cavity being set aside in the internal surface of said pad for the housing of the non invasive section of a catheter, and, on the other hand, a flexible fastening band or sheet, the central section of which is reattached to the external surface of the pad, the overflowing end portions of this flexible band fitted, on their internal surface, an adhesive coating designed to be applied on the skin of a patient, after removing the protective films.

This device also presents the same disadvantages that the devices mentioned above, namely:
it does not create, around the implantation port of the catheter, a waterproof barrier effectively opposing the passage of water and infectious agents in the direction of the insertion site of the catheter, due to the fact that the full area coated with self adhesive material of the pad may not match closely the reliefs and hollows (wrinkles) of the surface of the epidermis;
it may, for this same reason, peel off easily, unexpectedly, with complications that can arise from a wrong positioning of the catheter if we do not remedy the situation quickly.

A disadvantage common to all these devices is the result of the fact that glue is situated underside, during the provision of nursing care this does not allow a choice of skin area more appropriate for gluing. However this can prove inconvenient, for example, if the patient presents a wound in close proximity to the site of the catheter. In addition, if a catheter must stay in place several weeks or months, the devices being always glued to the same place, they sensitise the skin of the patient, which can create skin trauma or allergic reactions.

Another major drawback of all of these devices is that none of them provides a waterproof protection of the catheters.

An object of the invention is to remedy the disadvantages of holding and protection devices of catheters proposed at this time.

According to the invention, this goal is achieved thanks to maintaining necessary protection of catheters, comprising:
on the one hand, a device comprising:
a pad containing an internal face, and an external face opposite the internal face, the internal face being intended to be applied on the skin of a patient around a catheter, the pad having a cavity shaped to accommodate a non-invasive section (head or join) of catheter,
a thin and flexible covering film, the covering film having an integral central section of pad and an overflowing and non-glued surrounding fastening section that extends toward the outside from said central section, around the pad, the external face of the pad being applied against an internal face of the covering film intended to be applied on the skin of the patient, and,
on the other hand, a package containing a waterproof and non-allergenic adhesive, this package containing an outlet port shaped to allow the fitting of an adhesive bead linking the skin of the patient and the internal face of the cover sheet, the adhesive bead coming into contact with the surrounding section of fixing of the covering film.

The adhesive can be a liquid, a foam or preferably a gel adhesive. In the following description it thus makes reference to a gel adhesive, although other forms of adhesive are not excluded.

Through this, the application of the overflowing surrounding section of the covering film on the bead of adhesive gel, allows to create a close and waterproof junction between the skin and said cover sheet, thereby forming an effective means opposing any movement of the catheter. The holding device and protection of the catheters according to the invention provides several interesting advantages, particularly:
to allow firm hold of the catheters. In effect, the deposit of a continuous bead of adhesive between the skin and the protection sheet allows an effective fixing of the latter. This continuous bead placed in direct contact between the skin and the film totally guarantees total surrounding fastening, without problem of continuity;
to allow waterproof protection of the catheters. In effect, the deposit of a bead of continuous adhesive between the skin and the protection film also allows to ensure a perfect waterproof barrier.

This continuous barrier placed in direct contact between the skin and the film guarantees an excellent sealing, thus ensuring a guarantee against the risks of dirt or contamination of the protected sections, by contact with impure elements, including polluted liquids. For a good seal, the bead of adhesive gel is achieved preferably all around the pad, i.e. in surrounding the pad. The bead of surrounding gel thus also replacing the non-invasive section of the catheter, without making contact with this site,
allows “tailor-made” protection of the catheters where the site is generally very varied, by the gluing choice adapted to each situation;
to allow easy access for the inspection of the catheter or the replacement of the device, not requiring systematic removal of the catheter;
to be easy and fast to use;
to also be very economic.

The attached surrounding section of cover film, that is to say the section that extends around the pad, preferably overflows around the pad. A large attached section enables indeed to choose more freely on the position of the bead of adhesive gel. It is thus possible, where appropriate, to bypass an injured section or modify the skin of the patient, if such an injured or modified section is located in the vicinity of the catheter.

The fact that the attached surrounding section of covering film is not glued, gives it better permeability and allows skin to breathe in the covered area. The bead of gel certainly reduces the permeability of the covering film in the area where it is applied. This effect is, however, limited due to the local nature of the adhesive gel bead. In effect the bead of gel is preferably applied locally on surrounding section and not overlapping a limited surface.

Moreover, the bead of gel is preferably applied so as not to come into contact with the pad. However, it is possible to put a thin layer of adhesive gel on the internal face of the pad to facilitate its application on the skin.

According to one important characteristic of the device, the adhesive can present the properties of adhesion to the skin weaker than its properties of adhesion to the covering film, so as to allow the withdrawal of the device without painful effect or residues of adhesive on the skin.

According to a preferred exemplary embodiment, the pad presents a generally flat shape.

According to an embodiment, the device contains a detachable protection film applied against the external face of the cover film, this protection film presenting a lower flexibility than that of said cover film.

According to an exemplary embodiment, the covering film dimensions allow its cutting, this overall film being, preferably, executed in a non-allergenic material and easily divided into pieces or of smaller dimensions, for example by means of a pair of scissors.

According to an exemplary embodiment, the covering film is executed in polyurethane or polymer based polyurethane.

According to an exemplary embodiment, the covering film has a thickness between 2 micrometers and 3 millimeters, and preferably a thickness between 10 microns and 80 microns,

According to an embodiment, the pad is produced in any material with the robustness and flexibility necessary for firm support of the catheter and good comfort for the patient such as viscose, silicone, polypropylene, sodium polyacrylate, cellulose.

According to an exemplary embodiment, the adhesive is constituted by a copolymer, preferably on base of silicone, for example a glue base of polydimethylsiloxane, with functional groups, or polyacrylate base or acrylic base.

The objects, features and benefits as above, and still others, are shown better in the description that follows and annexed drawings in which:

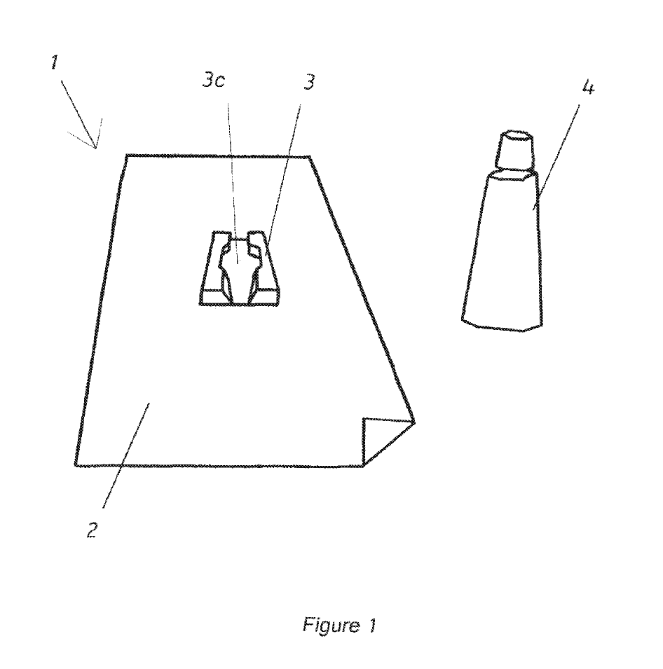

FIG. 1 is a perspective view of the elements constituting the kit where necessary according to the invention.

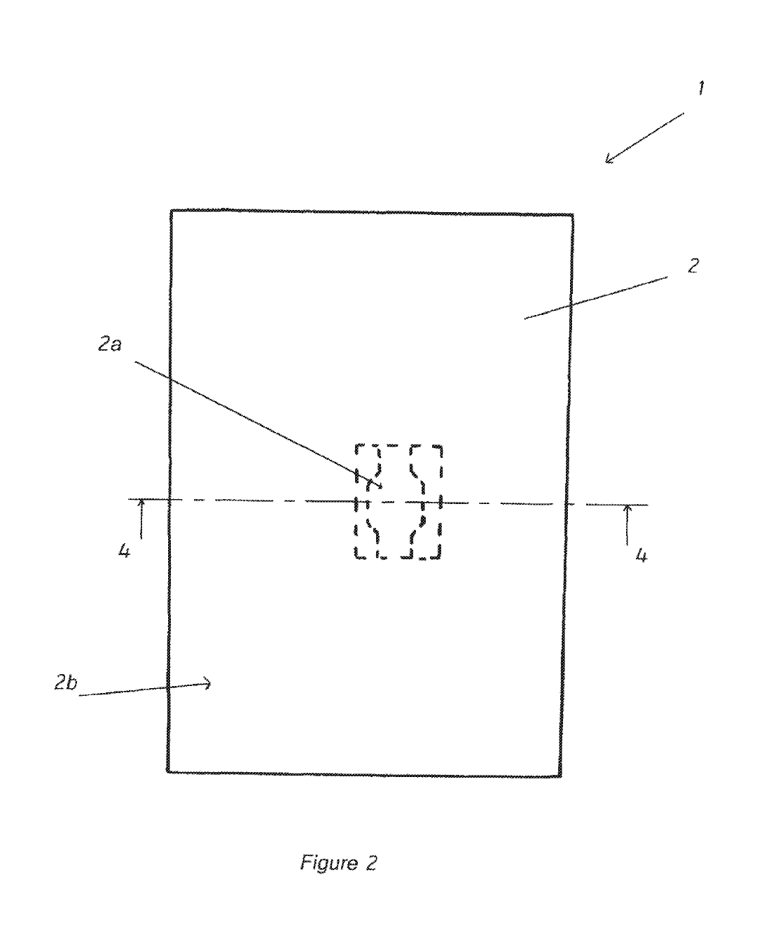

FIG. 2 is a top view of the holding device.

FIG. 3 is a bottom view of the latter.

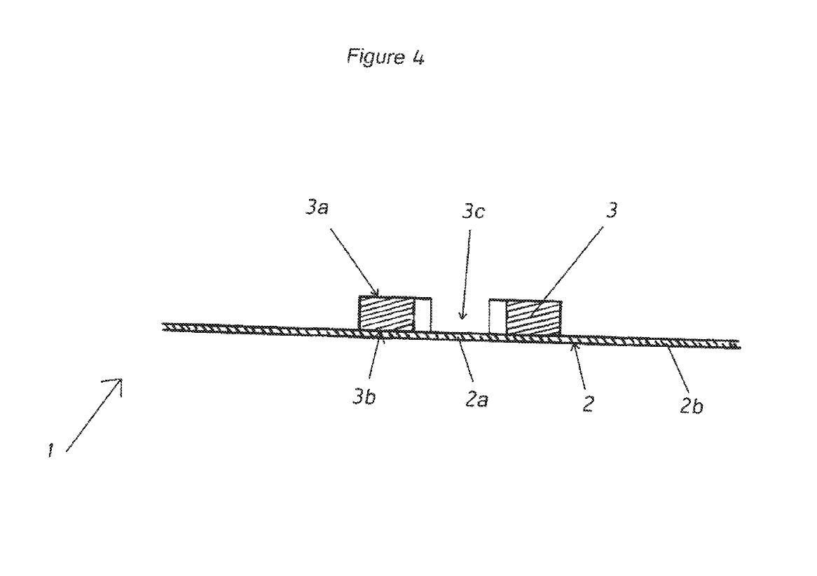

FIG. 4 is a sectional view and on a greater scale, according to line 4-4 of FIG. 2.

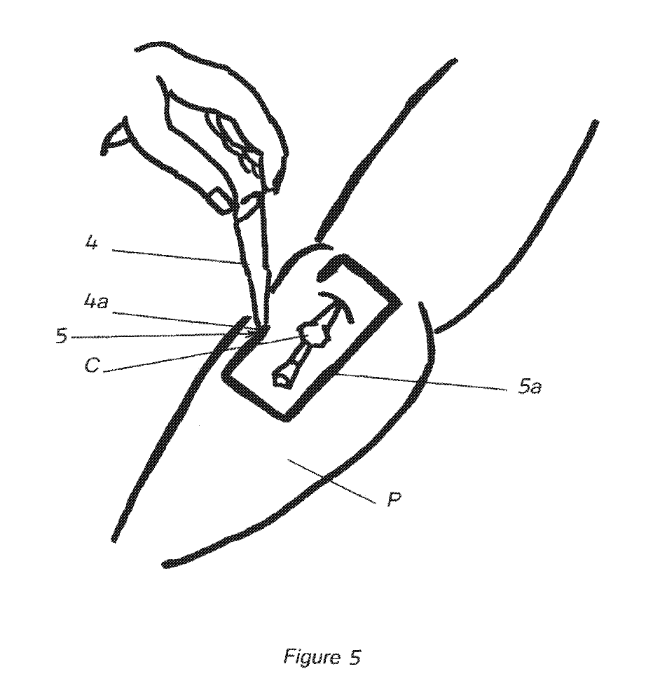

FIG. 5 is a perspective view showing a catheter inserted into a blood vessel of the forearm of a person and the positioning of a bead of self-adhesive gel around the non-invasive section of the catheter.

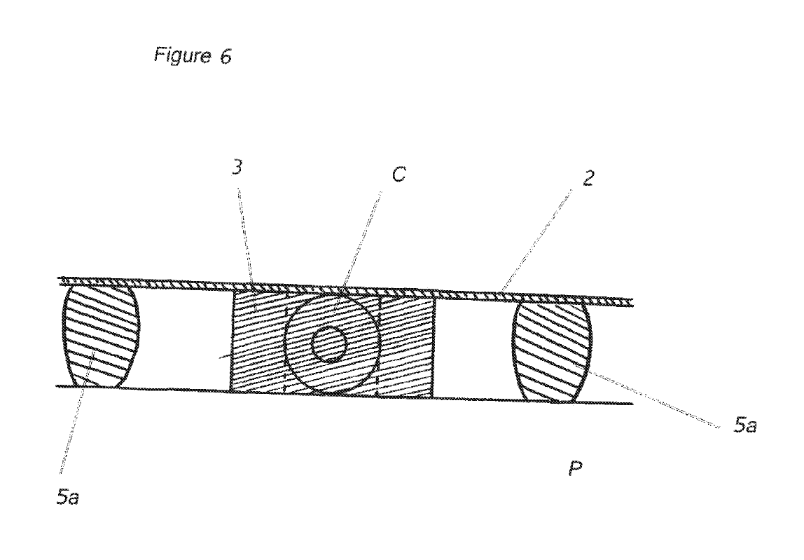

FIG. 6 is a sectional view and larger scale showing the attached device and the maintenance of the catheter on the forearm of a person.

We refer to with said drawings to describe an interesting example, but by no means limited to, realisation of the need for protection and maintenance of catheters, according to the invention.

The necessary protection and maintenance of catheters according to the invention includes essentially:
a device 1 consisting of a thin and flexible covering film 2 in which central section 2a is linked to a pad 3 shaped to accommodate the non-invasive section (head or join) of a catheter C put in place on an area of the body of a patient, and in which surrounding overflowing portion extends toward the outside from said central section 2a, still called the surrounding attached edge 2b, is not glued; and
a package 4 containing an adhesive gel 5.

The covering film 2 can be executed in any thin and flexible material and, preferably, in a non-allergenic material, for example in polyurethane or polymer based polyurethane, water and bacteria proof, and easily cut using a manual instrument such as a pair of scissors. Presents dimensions for cutting around the pad 3 appropriate to the shape and the site of the catheters. For example, it may present a shorter length to 20 cm and a width greater than 4 cm. It can be transparent, translucent, opaque, white or tinted, even decorated with various patterns.

Advantageously the device 1 still contains a detachable protection film (non-represented) applied against the external face of the covering film 2, this protection sheet presenting a lower flexibility than that of said covering sheet.

The internal face (face wherein located on the pad 3) of the surrounding edge 2b of the covering film 2 is intended to be applied on the skin P of the patient via a bead of adhesive gel.

On the other hand, it presents, advantageously, a thickness between 2 microns and 3 mm, and preferably, a thickness between 10 μm and 80 μm.

The pad 3 has an internal face 3a and one external face 3b. The internal face of the pad is intended to be applied on the skin P of a patient. It can be glued to the skin, or preferably, simply supported against the skin, thanks to the covering film. The pad presents a cavity 3c shaped to accommodate the non-invasive (head or join) of a catheter C. The external face of the pad is attached to the central section 2a of the covering film 2, by any suitable means known per se, by means of a biocompatible adhesive. Preferably, the pad 3 presents a generally flat shape.

As previously, the external face of the pad is applied against the internal face of the covering film. It can be glued or welded onto the covering film. It is also possible to use a small amount of adhesive gel to stick the pad on the covering film, in particular, presenting the necessary protection in which the provided pad being separated from the covering film.

The pad is produced in any material with the robustness and flexibility necessary for firm holding of the catheter C and of good comfort for the patient. For example, it is realised in viscose, silicone, polypropylene, sodium polyacrylate, cellulose.

The container of adhesive 4 may consist of a compressible tube containing an adhesive paste consistency 5. Of course, any other type of container would be usable. The container 4 comprises, preferably, the head of an extrusion or a neck 4a in accordance with, in a manner known per se, to allow the distribution of the adhesive 5 contents in said container in the form of a flexible bead 5a.

The adhesive 5 is also non-allergenic and biocompatible. It can be advantageously formed of by a copolymer, preferably on a silicone base, for example by a glue base of polydimethylsiloxane, with functional groups, or polyacrylate or acrylic base. The adhesive contents 5 in the container 4 is waterproof and has properties that allow to simultaneously adhere the skin P of the patient and to the covering film 2, without having been previously glued to one or the other of the two surfaces.

Preferably and advantageously, the adhesive 5 presents the properties of adhesion to the skin P of the patient less strong than its properties of adhesion to the covering film 2, so as to allow the withdrawal of device 1 without pain or residues of adhesive on the skin.

The adhesive 5 and the covering film 2 show the properties that make the elements perfectly compatible in adhesion of one to the other in position and respond to the constraints. In other words, the adhesive 5 and the covering film 2 are made in materials selected for their properties so that they can adhere to one another in such a way as to ensure the necessary sealing.

According to the implementation embodiment of necessary maintenance and protection according to the invention, the catheter is inserted in a lumen of a cavity of the body or of a blood vessel of a patient, to the desired location on the basis of its use; a bead 5a of flexible waterproof and non-allergenic adhesive, and presenting in addition of properties which enable it to adhere to both the skin and the protection sheet, is deposited on the best area of the skin P of the patient, around the location of the non-invasive (head or join) of the catheter C to protect and maintain, the adhesive gel 5 immerses into the wrinkles of the skin thus achieving a waterproof barrier against the risk of dirt or contamination by contact with impure elements, including polluted liquids.

The holding device 1 is then placed on the patient covering the catheter, so, on the one hand, matching the length of cavity 3c of housing the catheter C with the non-invasive section of said catheter C thus last realising close maintenance of this and, on the other hand, that the surrounding overflowing border 2B of the covering film 2, thin, flexible, waterproof and non-glued, preferably carried out in non-allergenic material, lies on the surrounding bead of adhesive 5a, thus realising a close junction between the skin and the individual covering film 2 and the waterproof insulation of catheter C, thus offering a close junction and seal between the skin P and said covering film 2, constituting an effective means opposing any movement of the catheter C.

The portion of edge 2b extending beyond the surrounding bead of adhesive gel 5a by relationship to catheter C can be cut to the desired dimensions and shapes, depending on the size, the shape and location of catheter C.

The protection and support device according to the invention thus applied on the skin of a patient can stay in place several days.

When device 1 must be replaced, for example for the inspection of the catheter, the covering film is removed by pulling on this last. The bonding connection torque between the adhesive 5 and the skin P of the patient being less important than the bonding torque of connection between the adhesive 5 and the covering film 2, the device 1 can be removed without pain or traumatic effect for the patient, and without leaving adhesive residue on the skin of the latter.

When positioning a new device 1, a new bead 5a of adhesive gel 5 is deposited on the best area of the skin P of the patient, around the location of the non-invasive section (head or fitting) of the catheter C, but varying slightly the gluing area compared to the previous gluing area.

The gluing areas can thus be alternated at each replacement of the device 1 in order to avoid risks of allergic reactions or possible trauma to skin that could occur with adhesive gel too long on same surface of skin.

1. A kit for the protection and maintenance of catheters, characterised in that it comprises:
on the one hand,
a pad (3) including an internal face (3a) and external face (3b), opposite to the internal face, the internal face of the pad is intended to be applied onto the skin (P) of a patient around a catheter (C), the pad with a cavity (3c) shaped for non-invasive section (head or fitting) of catheter,
a thin and flexible covering film, the covering film with a central section (2a) integral of the pad and surrounding overflowing section attached (2b) and not glued, that extends outwards from said central section, around the pad, the external face of the pad (3b) being applied against an internal face of the covering film intended to be applied on the skin of the patient, and,
on the other hand,
a package (4) containing an adhesive (5) which is waterproof and non-allergenic, this package with an outlet port shaped to allow positioning of a bead (5a) of the adhesive connecting locally to the skin of the patient and the internal face of the covering film, the bead (5a) of the adhesive coming into contact with the surrounding section of attached covering film.

2. A kit for the protection and maintenance of catheters according to claim 1, characterised in that the adhesive (5) presents properties of adhesion to the skin (P), less strong than its properties of adhesion to the covering film (2), to enable the removal of the device (1) without painful or traumatic effect for the patient, or residues of adhesive on the skin of the latter.

3. A kit for the protection and maintenance of catheters according to one of claim 1, characterised in that the adhesive (5) and the covering film (2) present properties that make the elements perfectly compatible in adhesion to be able to adhere to one another in position and to respond to the constraints, that is to say that the adhesive (5) and the covering film (2) are made in materials chosen for their properties so that they can adhere to one another in such a way as to ensure the necessary sealing.

4. A kit for the protection and maintenance of catheters according any one of claim 1, characterised in that the covering film (2) is made of polyurethane, or polymer based polyurethane.

5. A kit for the protection and maintenance of catheters according to any of the claim 1, characterised in that the pad (3) presents a shape generally flat.

6. A kit for the protection and maintenance of catheters according to any of the claim 1, characterised in that the device (1) includes a film of detachable protection applied against the external face of the covering film (2), this protection film presenting a flexibility lower than that of said covering film.

7. A kit for the protection and maintenance of catheters according to any of the claim 1, characterised in that the covering film (2) presents dimensions allowing cutting, overall this sheet being, preferably, executed in a non-allergenic material and easily divided into pieces or smaller dimensions, for example by means of a pair of scissors.

8. A kit for the protection and maintenance of catheters according to any one of claim 1, characterised in that the covering film (2) presents a thickness between 2 μm and 3 μm and, preferably a thickness of between 10 μm and 80 μm.

9. A kit for the protection and maintenance of catheters according to any one of claims claim 1, characterised in that the pad (3) is executed in any material with the robustness and flexibility necessary for firm holding of the catheter and good comfort for the patient, such as viscose, silicone, polypropylene, sodium polyacrylate, cellulose.

10. A kit for the protection and maintenance of catheters according to any of the claim 1, characterised in that the adhesive (5) consists of a copolymer with base of silicone, or polyacrylate or acrylic base.
